(19) United States
(12) Patent Application Publication
(10) Pub. No.: US 2015/0037844 A1
(43) Pub. Date: Feb. 5, 2015

(54) LIQUEFACTION AND SACCHARIFICATION OF GRANULAR STARCH AT HIGH CONCENTRATION

(71) Applicant: Danisco US Inc., Palo Alto, CA (US)

(72) Inventors: Sung Ho Lee, North Liberty, IA (US); Jayarama K. Shetty, Pleasanton, CA (US); Bruce A. Strohm, Beloit, WI (US)

(21) Appl. No.: 14/225,987

(22) PCT Filed: Sep. 7, 2012
(86) PCT No.: PCT/US2012/054190

§ 371 Date: Oct. 14, 2014

Related U.S. Application Data
(60) Provisional application No. 61/541,031, filed on Sep. 29, 2011.

(51) Int. Cl. C12P 19/20; C12P 19/02

(52) U.S. Cl. CPC C12P 19/20; C12P 19/02
USPC 435/96

(57) ABSTRACT

The present teachings provide methods of processing granular starch in slurries containing high dry solids content. The slurries are initially incubated with enzymes at or below the gelatinization temperature. The use of pullulanase and glucoamylase at specified doses allows for improved glucose yields at lower energy cost.

### CROSS-REFERENCE TO RELATED APPLICATION

This application claims the benefit of U.S. Provisional Application No. 61/541,031, filed Sep. 29, 2011, which is incorporated by reference in its entirety.

### BACKGROUND

The conversion of insoluble granular starch to glucose or other soluble dextrins is an important large-scale process to obtain end-products, such as sugar sweeteners, specialty syrups, enzymes, proteins, alcohol (e.g., ethanol, butanol), organic acids (lactic acid, succinic acid, citric acid) and specialty biochemicals such as amino acids, (lysine, monosodium glutamate) and 1-3 propanediol and to the biofuels industry. The partial crystalline nature of starch granules imparts insolubility in cold water. Solubilization of starch granules in water requires a tremendous amount of heat energy to disrupt the crystalline structure. The more water used to solubilize the granules, the more energy is required to heat the water. More energy is also required to evaporate the water after subsequent saccharification.

Solubilization can be performed by direct or indirect heating systems, such as direct heating by steam injection. (See for example, Starch Chemistry and Technology, eds R. L. Whistler et al., 2^{nd} Ed., 1984 Academic Press Inc., Orlando, Fla. and Starch Conversion Technology, Eds. G. M. A. Van Beynum et al., Food Science and Technology Series, Marcel Dekker Inc., NY). A typical conventional starch liquefaction system delivers an aqueous starch slurry under high pressure to a direct steam injection cooker that raises the slurry temperature from about 35-40° C. to 107-110° C. The slurry generally contains a thermal-stable alpha amylase in which case the pH is adjusted to favor the alpha amylase. Granular starch resulting from wet milling usually has a dry solid content of 40 to 42%. The concentration is generally diluted to 32% to 35% dry solids before heating above the liquefaction temperature. Without this dilution and consequent reduction in viscosity the feed pumps of the high temperature jet-cooking unit operation system cannot handle the slurry.

An alternative to the above conventional process has been described in which problems of excessive viscosity are avoided by not heating the granular starch slurry above the liquefaction temperature (see, e.g., U.S. Pat. No. 7,618,795 and US 20050136525). Instead, the granular starch is solubilized by enzymatic hydrolysis below the liquefaction temperature. Such “low-temperature” systems have been reported to be able to process higher concentrations of dry solids than conventional systems (e.g., up to 45%). However, no-cook systems have the disadvantage that a relatively long incubation of about 24 hours or more at moderately elevated temperature is required for substantially complete solubilization. The longer incubation is itself associated with high energy costs.

Because of the large scale on which granular starch is processed, even seemingly small improvements in efficiency can have great economic advantage. However, the conversion process has already been extensively analyzed to identify and implement such improvements (see, e.g., Martin & Brumm at pp. 45-77 in “Starch Hydrolysis Products: Worldwide Technology, production and applications New York, VCH Publishers, Inc. 1992 and Luenser, Dev. in Ind. Microbiol. 24.79-96 (1993)).

### SUMMARY OF THE CLAIMED INVENTION

The invention provides methods of processing granular starch comprising: (a) contacting granular starch, water and one or more granular starch hydrolyzing enzymes including an alpha-amylase and/or a glucoamylase to produce slurry in which the concentration of dry solids is greater than 38% by weight, (b) incubating the slurry at a temperature above 40° C. and at or below the gelatinization temperature of the granular starch for at least five minutes to produce a composition in which the granular starch has been partially hydrolyzed into oligo- and/or mono-saccharides by the one or enzymes; and (c) raising and holding the temperature of the partially hydrolyzed composition above the gelatinization temperature of the granular starch to produce a liquefied composition.

Some methods further comprise (d) contacting the liquefied composition with pullulanase and glucoamylase and incubating to produce glucose. In some methods, the incubation in step (b) is for a sufficient time that the concentration of insoluble dry solids at the conclusion of step (b) is no more than 38% by weight. In some methods, 2-30% of the dry solids are soluble at the conclusion of step (b). In some methods, the percentage of dry solids is at least 39% throughout steps (a)-(d). In some methods, the concentration of dry solids is 39-45% by weight throughout steps (a)-(d). In some methods, the percentage of dry solids remains the same or increases between steps (a) and (d). In some methods, no more than 10% by weight water is added to the slurry throughout steps (b), (c) and (d). In some methods, no more water is added to the slurry throughout steps (b), (c) and (d). In some methods, the one or more enzymes includes an alpha amylase. In some methods, the alpha amylase is a Bacillus alpha amylase. In some methods, the alpha amylase is SPEZYME® AA, SPEZYME® XTRA®, SPEZYME® FRED, GYZME® G997, TERMAMYL®, 120-L, LC, SC, SUPRA or Fuelzyme®. In some methods the one or more enzymes includes at least two types of alpha amylase. In some methods, the alpha amylase is thermostable and remains active in step (c).

In some methods, the temperature in step (b) is 55-67° C. In some methods, the incubating in step (b) is for 5 min to four hours. In some methods, the temperature of step (c) is 90-110° C. In some methods, the composition is held at 90-110° C. for 5 min to 4 hours. In some methods, the composition is held at 100-110° C. for 5-20 minutes and at 90-100° C. for 1-2 hr. In some methods, the ratio of pullulanase to glucoamylase in step (d) is at least 9:1 by units. In some methods, step (d) is performed at a temperature of 40-80° C. In some methods, step (d) is performed for 20-150 hours. In some methods, the yield of glucose is at least 95% by weight granular starch. In some methods, the yield of glucose is 95-96% by weight granular starch. In some methods, the one or more enzymes include an alpha-amylase and the method further comprises deactivating the alpha amylase after step (c).

In some methods, the deactivation of the alpha amylase is by heat or acid treatment. In some methods, no acids or bases are added to change the pH after step (a). In some methods, the pH is between 4.9 and 5.5 throughout steps (b), (c) and (d). In some methods, no more than one evaporation step is performed to concentrate the glucose during or after step (d).

In some methods, the pullulanase in step (d) is from Bacillus and the glucoamylase is from Aspergillus niger or Humicola grisea. In some methods, the pullulanase and glucoamylase are provided as a blend.

In some methods, the granular starch is produced by wet milling. In some methods, the granular starch is granular starch of wheat, barley, corn, rye, rice, sorghum, legumes, cassava, millet, potato, sweet potato, or tapioca.

In some methods, the one or more enzyme are one or more alpha amylases and the method further comprises allowing the liquefied composition to cool, whereby the one or more alpha amylases of step (a) or one or more fresh alpha amylases hydrolyze starch in the liquefied composition to oligosaccharides producing a maltodextrin composition. In some methods, allowing the liquefied composition to cool comprises incubating the liquefied composition at a temperature above atmospheric temperature and below the temperature of step (c).

In some methods, the granular starch gelatinizes over a range of temperature and the temperature in step (b) is below the low end of the range.

### BRIEF DESCRIPTION OF THE FIGURES

FIG. 1: Flow diagram comparing of starch liquefaction process of the present invention

FIG. 2: Impact of dry substance at 60° C. on slurries containing no added alpha amylase.

FIG. 3: Effect of partial solubilization and hydrolysis of granular starch on the peak viscosity at gelatinization temperature using 45% dry solid corn starch substrate.

### DEFINITIONS

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Hale & Markham, Harper Collins Dictionary of Biology, Harper Perennial, NY (1991) provide the ordinary meaning of many of the terms describing the invention.

“Starch” refers to any material including complex polysaccharide carbohydrates of plants, including amylose and/or amylopectin with the formula (C₆H₁₀O₅)ₓ, with X being any number. In particular, the term refers to any plant-based material, such as for example, grains, grasses, tubers and roots and more specifically wheat, barley, corn, rye, rice, sorghum, legumes, cassava, millet, potato, sweet potato, and tapioca.

“Granular starch” refers to uncooked (raw) starch, which has not been subject to gelatinization.

“Starch gelatinization” means solubilization of starch molecules to form a viscous suspension.

“Gelatinization temperature” is the lowest temperature at which gelatinization of a starch containing substrate begins. The exact temperature of gelatinization depends on the specific starch and may vary depending on factors such as plant species and environmental and growth conditions. The initial starch gelatinization temperature ranges for a number of granular starches which may be used in accordance with the processes herein include barley (52-59° C.), wheat (58-64° C.), rye (57-70° C.), corn (62-72° C.), high amylose corn (67-80° C.), rice (68-77° C.), sorghum (68-77° C.), potato (58-68° C.), tapioca (59-69° C.) and sweet potato (58-72° C.) (Swinkels, pg. 32-38 in STARCH CONVERSION TECHNOLOGY, Eds Van Beynum et al., (1985) Marcel Dekker Inc. New York and The Alcohol Textbook 3.sup.rd ED. A Reference for the Beverage, Fuel and Industrial Alcohol Industries, Eds Jacques et al., (1999) Nottingham University Press, UK). Gelatinization involves melting of crystalline areas, hydration of molecules and irreversible swelling of granules. The gelatinization temperature occurs in a range for a given grain because crystalline regions vary in size and/or degree of molecular order or crystalline perfection. STARCH HYDROLYSIS PRODUCTS Worldwide Technology, Production, and Applications (eds/Shenck and Hebeda, VCH Publishers, Inc, New York, 1992) at p. 26.

“DE” or “dextrose equivalent” is an industry standard for the concentration of total reducing sugars, and is expressed as % D-glucose on a dry weight basis. Unhydrolyzed granular starch has a DE that is essentially 0 and D-glucose has a DE of 100.

“Glucose syrup” refers to an aqueous composition containing glucose solids. Glucose syrup has a DE of more than 20. Some glucose syrup contain no more than 21% water and no less than 25% reducing sugar calculated as dextrose. Some glucose syrups include at least 90% D-glucose or at least 95% D-glucose. Sometimes the terms glucose and glucose syrup are used interchangeably.

“Hydrolysis of starch” is the cleavage of glucosidic bonds with the addition of water molecules.

A “slurry” is an aqueous mixture containing insoluble starch granules in water.

The term “total sugar content” refers to the total sugar content present in a starch composition including monosaccharides, oligosaccharides and polysaccharides.

The term “dry solids” (ds) refer to dry solids dissolved in water, dry solids dispersed in water or a combination of both. Dry solids thus include granular starch, and its hydrolysis products, including glucose.

“Dry solid” content refers to the percentage of dry solids both dissolved and dispersed as a percentage by weight with respect to the water in which the dry solids are dispersed and/or dissolved. The initial dry solid content is the weight of granular starch corrected for moisture content over the weight of granular starch plus weight of water. Subsequent dry solid content can be determined from the initial content adjusted for any water added or lost and for chemical gain. Subsequent dissolved dry solid content can be measured from refractive index as indicated below.

The term “high DS” refers to aqueous starch slurry containing dry solids greater than 38% by weight of dry solids plus water.

“Dry substance starch” refers to the dry starch content of granular starch and can be determined by subtracting from the mass of granular starch any contribution of water. For example, if granular starch has a water content of 20%, then 100 kg of granular starch has a dry starch content of 80 kg. Dry substance starch can be used in determining how many units of enzymes to use.

“Refractive Index Dry Substance” (RIDS) is the determination of the refractive index of a starch solution at a known DE at a controlled temperature then converting the RI to dry substance using an appropriate relationship, such as the Critical Data Tables of the Corn Refiners Association

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP4⁺ (>DP3) denotes polymers with a degree of polymerization of greater than 3.

“Contacting” refers to the placing of one or more enzymes and/or other reaction components in sufficiently close proximity to a substrate to enable the enzyme(s) to convert the substrate to an end product. Contacting can be effecting by combining or mixing solutions of the enzyme with the respective substrates.

“Enzyme activity” refers to the action of an enzyme on its substrate. “Hydrolysis of starch” refers to the cleavage of glucosidic bonds with the addition of water molecules.

An “alpha-amylase (E.C. class 3.2.1.1)” is an enzyme that catalyze the hydrolysis of alpha-1,4-glucosidic linkages. These enzymes have also been described as those effecting the exo or endohydrolysis of 1,4-α-D-glucosidic linkages in polysaccharides containing 1,4-α-linked D-glucose units. Another term used to describe these enzymes is glycogenase. Exemplary enzymes include alpha-1,4-glucan 4-glucanohydrase glucanohydrolase.

A “glucoamylase” refers to an amyloglucosidase class of enzymes (EC.3.2.1.3, glucoamylase, alpha-1,4-D-glucan glucohydrolase) are enzymes that remove successive glucose units from the non-reducing ends of starch. The enzyme can hydrolyze both linear and branched glucosidic linkages of starch, amylose and amylopectin. The enzymes also hydrolyze alpha-1, 6 and alpha-1, 3 linkages although at much slower rates than alpha-1, 4 linkages.

“Pullulanase” also called debranching enzyme (E.C. 3.2.1.41, pullulan 6-glucanohydrolase), is capable of hydrolyzing alpha 1-6 glucosidic linkages in an amylopectin molecule.

A “Liquefon Unit” (LU) is a measure of the digestion time required to produce a color change with iodine solution, indicating a definite stage of dextrinization, Example 1 of starch substrate under specified conditions (see, e.g., U.S. Pat. No. 5,756,714).

An “end product” is any carbon-source derived molecule product which is enzymatically converted from the granular starch substrate. Preferably, the end product is glucose or glucose syrup. Glucose can be used as a precursor for other desired end-products.

“Yield” refers to the amount of a desired end-product/products (e.g., glucose) as a percentage by dry weight of the starting granular starch.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over the length of the shorter sequence (if lengths are unequal), determining the number of positions at which the identical residues occurs in both sequences to yield the number of matched positions, dividing the number of matched positions by the total number of matched and mismatched positions not counting gaps, and multiplying the result by 100 to yield the percentage of sequence identity.

The term “comprising” and its cognates are used in their inclusive sense; that is, equivalent to the term “including” and its corresponding cognates.

Numeric ranges are inclusive of the numbers defining the range. Some preferred subranges are also listed, but in any case, reference to a range includes all subranges defined by integers included within a range.

### DETAILED DESCRIPTION

### I. General

The invention provides methods of processing granular starch. The invention is based in part on the result that pretreatment of granular starch with one or more granular starch hydrolyzing enzymes at a relatively low temperature substantially reduces the subsequent viscosity and dilatancy (shear thickening) when the temperature is subsequently raised above the gelatinization temperature and the granular starch solubilizes to become liquefact. The pretreament results in significantly more granular starch dissolving than would occur in a conventional process, in which after addition of enzymes, the temperature of granular starch is immediately raised above the gelatinization temperature. However, unlike processes conducted at low temperature throughout, the pretreatment leaves much granular starch undissolved. Surprisingly significant reduction in viscosity can be obtained notwithstanding the ultimate amount of dissolved solids is unchanged. Even a small amount of pretreatment allows granular starch to be processed at an initial concentration higher than 38% dry solids using current industry equipment. Thus, with the present methods, granular starch received from wet mills at a typical concentration of 40-42% dry solids, can be processed as is on conventional equipment rather than being diluted first. Processing at increased concentration reduces the amount of water, heat energy and reagents required to process a given amount of granular starch. For large scale-processing of granular starch, the savings have great practical significance. The higher concentration of starch processable by the present methods also has advantages for downstream saccharification in that the same or better yields of glucose can be obtained with less processing (e.g., evaporation steps). Using an appropriate blend of pullulanase and glucoamylase, a glucose yield of 95% or greater can be achieved from an initial concentration of dry solids of greater than 38%.

### II. Starting Material

The starting material for the process is granular starch. Plant material comprising granular starch may be obtained from sources such as wheat, corn, rye, sorghum (milo), rice, millet, barley, triticale, cassava (tapioca), potato, sweet potato, sugar beets, sugarcane, and legumes such as soybean and peas. Preferred plant material includes corn, barley, wheat, rice, milo and combinations thereof. Plant material may include hybrid varieties and genetically modified varieties (e.g. transgenic corn, barley or soybeans comprising heterologous genes). Any part of the plant may be used to as plant material including plant parts such as leaves, stems, hulls, husks, tubers, cobs, grains and the like. Whole grain can also be used as a source of granular starch. Preferred whole grains include corn, wheat, rye, barley, sorghum and combinations thereof. Preferably the whole grain is reduced in size by techniques such as milling (e.g. hammer milling or roller milling); emulsion technology; rotary pulsation; fractionation and the like.

The granular starch is preferably produced by wet milling of corn. A typical wet milling process starts with dried corn kernels that are inspected and cleaned to remove the cobs, chaff and other debris. The corn is then soaked in large tanks with small amounts of sulfur dioxide and lactic acid. These two chemicals, in warm water help soften the corn kernel over a 24-48 hour steeping period. During this time, the corn swells and softens and the mild acid conditions loosen the gluten bonds to release the starch. After steeping, the corn is coarsely ground. the ground corn and some steep water are passed through a separator, which essentially allows the germ, or the lightweight oil-containing portion, to float to the top of the mixture and be removed. The fibrous material is screened off, and granular starch and protein are separated by density using large centrifuges. The granular starch is often provided at a concentration of about 40-42% granular starch by dry weight. The concentration can be used as is in the process below or can be adjusted by dilution or centrifugation of filtration to give any desired concentration over 38% by dry weight.

Granular starch also includes grain flours from dry mills including ground whole grain or various fractions purified by the removal of non-starch fractions such as pericarp and germ and proteins.

### III. The Conversion Process

A slurry is formed by contacting granular starch, water and one or more granular starch hydrolyzing enzymes. The components of the slurry can be combined in any order. Preferably, the water and granular starch are combined first followed by addition of the enzyme(s). The water is typically normal tap water, but can any kind of water (e.g., water directly from a natural source, recycled water such as condensate from evaporation, or distilled water). The water can be supplied heated to at or near the intended incubation temperature, or at any other temperature (e.g., atmospheric temperature), in which case heat can be supplied to bring the slurry to the intended incubation temperature. Small amounts e.g., less than 1:100 by weight of additives to weight of water, such as acids, bases, salts or other excipients can be combined in the slurry to adjust the pH or otherwise improve enzyme activity. Such components can be added either as a component of the water or otherwise combined into the slurry. The pH is preferably in a range of 4-6.5 and more preferably 4.9-5.5.

When the granular starch is first combined into the slurry the concentration of granular starch measured as a weight dry solids:weight water plus dry solids in the slurry is greater than 38%. Here, as elsewhere in this application, the actual weight of granular starch introduced into a slurry is corrected for its moisture content, which is often around 11%. For example, if 45 g granular starch with a moisture content of is combined with 55 g water, the concentration of dry solids is 45×0.89/100=40.05%. No correction is made in calculating the percentage of dry solids for any non-carbohydrates components of granular starch other than water. Although some protein and lipids may be present, their weight is negligible compared with that of carbohydrates.

Optionally, the initial concentration of granular starch by percent dry solids is at least 38.5, 39,40, 41, 42, 43, 44, 45, 46, 47, 48, 49, or 50% and optionally is also less than 65%, 60%, 55% or 50% including all permutations of upper and lower limits. For example, the initial concentration is sometimes, 39-50%, 39-45%, 40-50% or 40-45% or 41-50% or 41-45%, or 42-50% or 42-45%. Preferably, the initial concentration is 38.5-42, 38.5-43, 39-42, 39-43. The weight of enzymes or any other minor components of the slurry is usually negligible and need not be taken into account in determining the percentage of granular starch by weight of water. Initially, essentially none of the granular starch is dissolved in the water. But as the granular starch processing enzyme(s) act on the granular starch, the starch is partially hydrolyzed to mono and oligosaccharide. Mono and oligosaccharides are water soluble and dissolve.

The quantity of enzymes depends on the type of enzyme and its activity. In general, an amount of about 0.01 to 5.0 kg of the thermostable alpha amylase is added to a metric ton (MT) dry solids of the raw material, preferably about 0.5 to 2.0 kg dry solids or about 0.1 to 1.0 kg dry solids. Glucoamylase if used can be provided at the same weight ranges as stated for alpha amylase. For example, generally an amount of between about 0.01 to 1.0 kg of SPEZYME® XTRA and SPEZYME® FRED (Danisco-Genencor) or their variants is added to a metric ton of dry solids of starch. For example, the enzyme can be added in an amount between about 0.05 to 1.0 kg dry solids; between about 0.1 to 0.6 kg dry solids; between about 0.2 to 0.6 kg and between about 0.4 to 0.6 kg of SPEZYME® XTRA and SPEZYME® FRED per metric ton of dry solids starch.

The slurry can be subject to agitation to increase dispersion of the granular starch in the water and facilitate action of the granular starch processing enzymes.

The temperature at which the slurry in incubated is selected to facilitate activity of the granular starch processing enzymes in partially hydrolyzing granular starch but not result in substantial if any liquefaction of granular starch other than by dissolving of hydrolysis products. Such can be achieved by using a temperature above room temperature and usually above 40° C. and not substantially greater than the liquefaction temperature of the granular starch. Preferably, the temperature is above 40° C. and at or below the gelatinization temperature of the granular starch. The gelatinization temperature of the granular starch may vary depending on the preparation and source but is usually within a range of 52-80° C. For many given source materials, the gelatination temperature can be expressed as a sub-range within 52-80° C. (see exemplary ranges provided in Definitions). In this case, the incubation temperature is preferably at or below the lower limit of the sub-range, or can be at or below the mid-point or the upper limit of the sub-range. Preferably, the incubation temperature is below the lower point of the temperature range in which gelatinization occurs for a given source of granular starch.

The one or more enzymes with granular starch hydrolyzing activity include an alpha-amylase and/or a glucoamylase. Inclusion of an alpha-amylase is preferred. If conversion to maltodextrins is desired, it is preferably that no enzyme other than an alpha amylase is used. The alpha amylase is preferably thermostable such that it remains active when the temperature of the slurry is raised above the gelatinization temperature. The one or more enzymes can include two enzymes of the same type (e.g., two alpha-amylases from different sources) in which case, the amounts of enzyme to add by mass or units apply to the blend of the enzymes. If more than one enzyme with granular starch processing activity is present, the enzymes can be supplied as a blend or separately.

The period for which the slurry is so incubated can depend on the concentration of granular starch and activity of the enzymes, among other factors. Other things being equal, the more concentrated the granular starch the longer the time, and the more active the enzymes, the shorter the time. The activity of the enzymes in turn depends on the amounts and types of enzyme(s) combined into the slurry and the temperature of incubation. The object of the incubation is to obtain sufficient partial hydrolysis and dissolving of the granular starch, such that the slurry can be liquefied without impractical increase in viscosity. However, excessive incubation and partial hydrolysis at this stage is unnecessary because further hydrolysis and dissolving occurs in the subsequent step when the temperature is raised up the gelatinization temperature. The incubation is preferably of sufficient time under the conditions employed that the concentration of starch remaining insoluble in water under the conditions of the incubation expressed as a percentage of the weight of water plus weight of granular starch initially provided is no more than 38% by weight. Alternatively, the incubation can be for sufficient time that at least 2, 3, 4, 5, 6, 7, 8, 9, 10, 15, or 20% and optionally up to 25% 30%, 40%, or 50%, of the dry solids of the granular starch has been rendered soluble in water under the conditions of the incubation, including all permutations of upper and lower limits. Preferably, the incubation renders 2-30 or 5-30% by weight of the granular starch soluble under the conditions of the incubation.

The period is usually at least five minutes, with 5 minutes to 4 hours being typical. For example, the slurry can be incubated for at least 10, 20, 30, 60, 120, or 180 minutes, and for no more than 4 hours. The slurry is sometimes incubated for 10 min to 2 hours or 20 min to 1 or 2 hr or 30 min to 1 or 2 hr. The incubation can also be for longer periods, e.g., up to 24 hr or up to 48 hr.

After sufficient partial hydrolysis and dissolving of granular starch has occurred, the temperature of the resulting partially hydrolyzed composition is raised and held above the gelatinization temperature of the granular starch. For a given source in which the gelatination temperature is expressed as a range (see above), the temperature is preferably held above the upper point of the range but can also be held above the midpoint of the range. The temperature can be rapidly raised by direct or indirect heating, for example, flowing the composition through a heated coil or by injection of steam. The temperature used in this step reflects a balance of several considerations. A higher temperature more rapidly liquefies granular starch. However, it is preferred that the temperature is not so high as to completely inactivate granular starch hydrolyzing activity. The hydrolysis of starch above the gelatinization temperature is sometimes referred to as dextrinization.

A heat-stable alpha amylase can remain active in this step with appropriate temperature selection. Thus, for example, the temperature is typically raised and held at a temperature of at least 80° C. degrees and more preferably at least 90° C. but usually no more than 120 or 110° C. for a period of at least 5, 10, 30 or 60 min and usually no more than 3 or 4 hours. For example, the temperature can be raised and held within a range of 90-110° C. for a period of 5 min to 4 hours, 5-120 min, 5-60 min, 10-180 min, 10-120 min, 10-60 min, 20-180 min, 20-120 min or 20-60 min. Sometimes, the temperature is raised and held at 100-110° C. for 5-20 minutes and then reduced to 90-100° C. for 60-120 min. In this step, the granular starch continues to be partially hydrolyzed by one or more enzyme(s) assuming the enzyme(s) are still active and is also directly liquefied by dissolving into the water. The direct liquefaction without prior hydrolysis may somewhat increase the viscosity of the composition. However, as a result of the prior incubation with enzyme(s) before liquefaction, the increase in viscosity does not exceed manageable levels.

The incubation is preferably continued until the granular starch has substantially or completely liquefied (e.g., at least 95, 96, 97, 98. 98.5 or preferably 99% liquefaction).

Subsequent processing depends on the desired product. For production of a maltodextrin composition the liquefied composition is allowed to cool and optionally held at a temperature between that used for liquefaction and atmospheric temperature. Incubation at such a temperature allows further hydrolysis mediated by alpha-amylase. The alpha-amylase can be the enzyme(s) initially supplied, if still active, and/or can be freshly supplied enzymes. The maltodextrins generated have a monomer content of about 3-19 glucose units and a DE value of 3-20. Maltodextrins can be isolated as a dry powder by evaporation. Maltodextrins are used as an additive in many processed food.

Alternatively, for production of glucoses, any remaining alpha-amylase in the liquefied composition can be inactivated because the oligosaccharides resulting from alpha amylase action are less amenable to action of glucoamylase than longer starch molecules. The inactivation can be performed by raising the temperature over 110° C. or by acid treatment at lower temperature (e.g., reducing pH to 4.2 at 950 for 30 min).

The resulting composition with or without inactivation of residual alpha amylase activity is allowed to cool and combined with fresh enzymes to complete hydrolysis of the now liquefied starch to glucose. Optionally, the pH can be adjusted as appropriate for these enzymes. A pH of 5.5 to 6 is preferred for some enzymes. The enzymes combined with the composition include at least a pullulanase and a glucoamylase. The two enzymes are preferably present at a ratio of at least 9:1 pullulanase to glucoamylase by units. The ratio can be for example between 1:9 and 1:50 glucoamylase to pullulanase by units, for example between 1:9 and 1:20 or 1:9 and 1:15. The glucoamylase is preferably added at at least 0.08 GAU/g dry substance starch (gdss) solids, for example, within a range of 0.08-0.14 GAU/gdss. The units of pullulanase are calculated from the GAU units multiplied by a factor reflecting the desired ratio, e.g., a factor of 9 for a 1:9 ratio. After cooling from above liquefaction temperature, the composition is incubated at temperature appropriate for activity of the enzymes, typically 40-80° C., 50-70° C. or preferably 55-60° C. The incubation is continued until the yield of glucose as percentage by weight of starting granular starch is at least 90% and preferably at least 93% or at least 95% or more than 95%. The yield is preferably 93-96% (or more), and sometimes 95-96%. The incubation time to achieve such a yield can vary, e.g., at least 5, 20, or 20 hours and up to 100 or 150 hours.

After a satisfactory yield of glucose has been obtained, the concentration of glucose syrup can be increased by evaporating water. Because of the higher concentration of granular starch used initially, the concentration of dry solids of the glucose syrup is also usually higher than in previous methods. In consequence, an adequate concentration of glucose syrup can be obtained with no more than one evaporation step.

The above process for production of glucose can be conceptualized as four steps involving forming a slurry, partially hydrolyzing granular starch of the slurry using one or more enzymes at relatively low temperature, raising the temperature so granular starch liquefies, and then supplying fresh enzymes and completing conversion of the starch to glucose. As was indicated above, the initial concentration of granular starch can be above 38% by dry weight. Thereafter, the concentration of granular starch and its oligosaccharide and monosaccharide hydrolysis products (collectively dry solids) can remain at above 38% through the above steps without unmanageable increase in viscosity. In fact, the percentage of dry solids may increase because some water is chemically incorporated into solids by the hydrolysis process (known as chemical gain) and/or due to loss of water by evaporation. In some methods, the percentage of dried solids is more than 38, 38.5, 39, 40, 41, 42, 43, 44, 45, 46, 47, 48, 49 or 50% throughout the above steps. In some such methods, the percentage of dry solids is no more than 65, 60, 55 or 50% throughout the above steps. In some methods, the percentage of dried solids is at least 38.5, 39, 40, 41, 42, 43 or 44 and not more than 45, 50 or 55% including all permutations of upper and lower limits. Preferably, the percentage of dried solids is 38.5-45% or 39-45% throughout the above teps. With the possible exception of adding small quantities of acids or bases to adjust the pH, it is not usually necessary to add significant quantities of water (e.g., an aggregate increase of greater than 10% by weight of water already present) throughout the above steps. In some methods, no water is added after forming the slurry.

The overall efficiencies can thus exceed those of conventional processes in unmanageable viscosities are avoided either by not exceeding a concentration of 38% by weight granular starch or by not heating the granular starch above its gelatinization temperature, in which case a much longer and less efficient incubation with granular processing enzymes is needed.

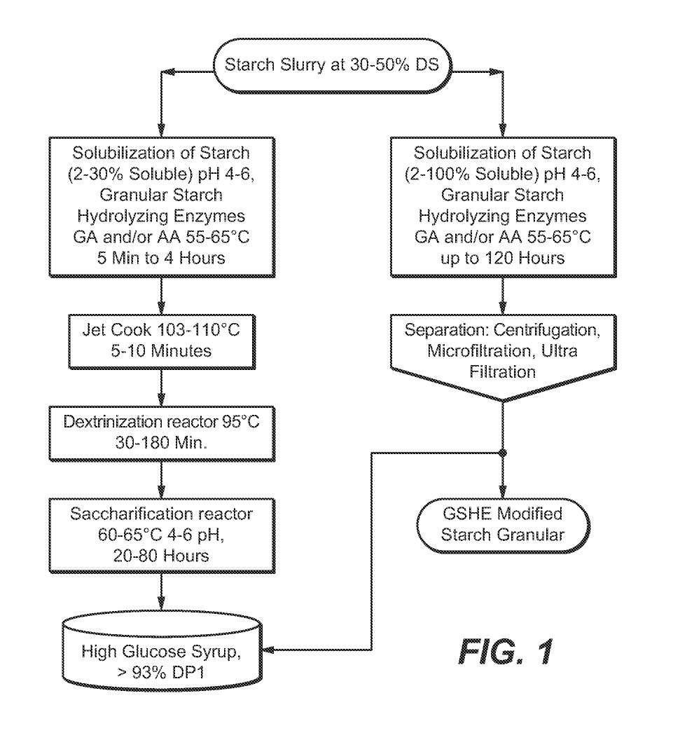

FIG. 1 compares an exemplary embodiment of the present methods with a no-cook method of hydrolyzing granular starch as previously reported. In each case, an initial slurry of granular starch in water is incubated with enzymes with granular starch hydrolyzing activity at moderate temperature (i.e., 55-65° C.) below the liquefaction temperature. However, the incubation is much shorter according to the present methods (e.g., 5 min to 4 hr, compared with 20-120 hr). In the present methods, the incubation can result in 2-30% of the granular starch liquefying in 5 min to 4 hours. The low-temperature method results in 2-100% liquefaction depending on the length of the incubation, an incubation of about 120 hours being required for near 100% solubility.

In the no-cook method, residual undissolved starch is removed by centrifugation and filtration. The undissolved granular starch can be recycled through the process again. The liquefied starch is subject to saccharification. In the present methods, the composition resulting from moderate temperature incubation then has the temperature raised above the liquefaction temperature, initially at 103-110° C. and then at 95° C. The heat liquefies the granular starch. Heat-stable alpha-amylase, if present, continues to hydrolyze the granular starch. The solubilized granular starch is then subject to saccharification.

### IV. Enzymes Having Granular Starch Hydrolyzing Activity

Enzymes having granular starch hydrolyzing activity (GSHEs) are able to hydrolyze granular starch. Such enzymes can be obtained from fungal, bacterial and plant cells such as Bacillus sp., Penicillium sp., Humicola sp., Trichoderma sp. Aspergillus sp. Mucor sp. and Rhizopus sp. These enzymes include enzymes having glucoamylase activity and/or alpha-amylase activity (See, Tosi et al, (1993) Can. J. Microbiol. 39:846-855).

Preferably, the GSHE(s) used in the present methods includes at least one alpha amylase. Alpha amylase is a microbial enzyme having an E.C. number, E.C. 3.2.1.1-3 and in particular E.C. 3.2.1.1. Preferably, the alpha amylase is a thermostable alpha amylase. Suitable alpha amylases may be naturally occurring as well as recombinant and mutant alpha amylases. Optionally, the alpha amylase is derived from a Bacillus species. Preferred Bacillus species include B. subtilis, B. stearothermophilus, B. lentus, B. licheniformis, B. coagulans, and B. amyloliquefaciens (U.S. Pat. No. 5,763,385; U.S. Pat. No. 5,824,532; U.S. Pat. No. 5,958,739; U.S. Pat. No. 6,008,026 and U.S. Pat. No. 6,361,809). Particularly preferred alpha amylases are derived from Bacillus strains B. stearothermophilus, B. amyloliquefaciens and B. licheniformis. Some preferred strains include having ATCC 39709; ATCC 11945; ATCC 6598; ATCC 6634; ATCC 8480; ATCC 9945A and NCIB 8059.

Another example of a GSHE having alpha-amylase activity is derived from a strain of Aspergillus such as a strain of A. awamori, A. niger, A. oryzae, or A. kawachi and particularly a strain of A. kawachi. Optionally, the A. kawachi enzyme having GSHE activity has at least 85%, 90%, 92%, 94%, 95%, 96%, 97%, 98% and 99% sequence identity to the amino acid sequence of SEQ ID NO: 3 of WO 05/118800 and WO 05/003311.

Commercially available alpha amylases suitable for use in the present methods include SPEZYME® AA, SPEZYME® XTRA, SPEZYME® FRED (acid-stable, low-Ca, thermostable, from Bacillus licheniformis), GZYME™ G997 (thermostable, non-genetically modified) (Genencor A Danisco Division) and TERMAMYL™ 120-L, LC, SC and SUPRA Bacillus licheniformis thermostabile alpha amylase, (Novozymes) and FUELZYME® LF (thermostable) (Verenium).

Alternatively or additionally, a GSHE having glucoamylase activity can be used. One such enzyme is derived from a strain of Humicola grisea, particularly a strain of Humicola grisea var. thermoidea (see, U.S. Pat. No. 4,618,579). Preferably, the Humicola enzyme having GSH activity has at least 85%, 90%, 92%, 94%, 95%, 96%, 97%, 98% or 99% sequence identity to the amino acid sequence of SEQ ID NO: 3 of WO 05/052148. Another example of a GSHE having glucoamylase activity is derived from a strain of Aspergillus awamori, particularly a strain of A. awamori var. kawachi. Optionally, the A. awamori var. kawachi enzyme having GSH activity has at least 85%, 90%, 92%, 94%, 95%, 96%, 97%, 98% and 99% sequence identity to the amino acid sequence of SEQ ID NO: 6 of WO 05/052148. Another example of a GSHE having glucoamylase activity is derived from a strain of Rhizopus, such as R. niveus or R. oryzae. The enzyme derived from the Koji strain R. niveus is sold under the trade name “CU CONC” or the enzyme from Rhizopus sold under the trade name GLUZYME. Another useful GSHE having glucoamylase activity is SPIRIZYME™ Plus (Novozymes A/S.

A Rhizopus oryzae GSHE with glucoamylase activity has been described in Ashikari et al., (1986) Agric. Biol. Chem. 50:957-964 and U.S. Pat. No. 4,863,864. A Humicola grisea GSHE mixture including a glucoamylase and potentiating activity has been described in Allison et al., (1992) Curr. Genet. 21:225-229; WO 05/052148 and European Patent No. 171218. An Aspergillus awamori var. kawachi GSHE has been described by Hayashida et al., (1989) Agric. Biol. Chem 53:923-929. An Aspergillus shirousami glucoamylase GSHE has been described by Shibuya et al., (1990) Agric. Biol. Chem. 54:1905-1914.

Enzymes having GSHE activity also include hybrid enzyme, for example one containing a catalytic domain of an alpha-amylase such as a catalytic domain of an Aspergillus niger alpha-amylase, an Aspergillus oryzae alpha-amylase or an Aspergillus kawachi alpha-amylase and a starch binding domain of a different fungal alpha-amylase or glucoamylase, such as an Aspergillus kawachi or a Humicola grisea starch binding domain. Alternatively, a hybrid enzyme having GSHE activity can include a catalytic domain of a glucoamylase, such as a catalytic domain of an Aspergillus sp., a Talaromyces sp., an Althea sp., a Trichoderma sp. or a Rhizopus sp. and a starch binding domain of a different glucoamylase or an alpha-amylase. Other hybrid enzymes having GSH activity are disclosed in WO 05/003311, WO 05/045018; Shibuya et al., (1992) Biosci. Biotech. Biochem 56: 1674-1675 and Cornett et al., (2003) Protein Engineering 16:521-520.

### V. Saccharification Enzymes

a. Glucoamylase

One or more glucoamylases (E.C. 3.2.1.3.) can be used as a saccharification enzyme (as well as or instead of use as liquefaction enzymes). The same or different glucoamylase can be used in saccharification as liquefaction. Whereas for liquefaction a glucoamylase should be active on granular starch, for saccharification a glucoamylase should be active on dissolved starch. The glucoamylase used for saccharification need not be active on granular starch. Suitable glucoamylases include those endogenously expressed by bacteria, plants, and/or fungi as well as recombinantly expressed glucoamylases heterologous to the host cells (e.g., bacteria, plants and/or fungi). Recombinantly expressed glucoamylases can be natural sequences, mutated sequences or hybrid sequences. Several strains of filamentous fungi and yeast produce suitable glucoamylases. For example, the commercially available glucoamylases produced by strains of Aspergillus and Trichoderma can be used. Hybrid glucoamylase include, for example, glucoamylases having a catalytic domain from a GA from one organism (e.g., Talaromyces GA) and a starch binding domain (SBD) from a different organism (e.g.; Trichoderma GA). A linker can be included with the starch binding domain (SBD) or the catalytic domain.

Examples of glucoamylases that can be used include Aspergillus niger G1 and G2 glucoamylase (See e.g., Boel et al., (1984) EMBO J. 3:1097-1102; WO 92/00381, WO 00/04136 and U.S. Pat. No. 6,352,851); Aspergillus awamori glucoamylases (See e.g., WO 84/02921); Aspergillus oryzae glucoamylases (see e.g., Hata et. al., (1991) Agric. Biol. Chem. 55:941-949) and Aspergillus shirousami (see e.g., Chen et. al., (1996) Prot. Eng. 9:499-505; Chen et al. (1995) Prot. Eng. 8:575-582; and Chen et al., (1994) Biochem J. 302:275-281). [083]. Other glucoamylases that can be used include those from strains of Talaromyces ((e.g., T. emersonii, T. leycettanus, T. duponti and T. thermophilus glucoamylases (See e.g., WO 99/28488; U.S. Pat. No. RE: 32,153; U.S. Pat. No. 4,587,215)); strains of Trichoderma, (e.g., T. reesei) and glucoamylases having at least about 80%, about 85%, about 90% and about 95% sequence identity to SEQ ID NO: 4 disclosed in US Pat. Pub. No. 2006-0094080; strains of Rhizopus, (e.g., R. Niveus and R. oryzae); strains of Mucor and strains of Humicola, ((e.g., H. grisea (See, e.g., Boel et al., (1984) EMBO J. 3:1097-1102; WO 92/00381; WO 00/04136; Chen et al., (1996) Prot. Eng. 9:499-505; Taylor. et al., (1978) Carbohydrate Res. 61:301-308; U.S. Pat. No. 4,514,496; U.S. Pat. No. 4,092,434; U.S. Pat. No. 4,618,579; Jensen et al., (1988) Can. J. Microbiol. 34:218-223 and SEQ ID NO: 3 of WO 2005/052148)). Optionally, the glucoamylase has at least about 85%, about 90%, about 92%, about 94%, about 95%, about 96%, about 97%, about 98% and about 99% sequence identity to the amino acid sequence of SEQ ID NO: 3 of WO 05/052148. Other glucoamylases that can be used include those obtained from Athelia rolfsii and variants thereof (See e.g., WO 04/111218) and Penicillium spp. (See e.g., Penicillium chrysogenum) and three forms of glucoamylase produced by a Rhizopus sp., namely “Gluc1” (MW 74,000), “Gluc2” (MW 58,600) and “Gluc3” (MW 61,400). Commercially available glucoamylases useful in the present methods include, for example, DISTILLASE® L-400, OPTIDEX® L-400 and G ZYME® G990 4X, GC480, G-ZYME 480, (Danisco US, Inc, Genencor Division) CU.CONC® (Shin Nihon Chemicals, Japan), GLUCZYME (extract of koji cultured wheat bran from R. Niveus)(Amano Pharmaceuticals, Japan (See e.g. Takahashi et al., (1985) J. Biochem. 98:663-671

### B. Pullulanase

These enzymes are generally secreted by a Bacillus species. For example Bacillus deramificans (U.S. Pat. No. 5,817,498; 1998), Bacillus acidopullulyticus (European Patent #0 063 909 and Bacillus naganoensis (U.S. Pat. No. 5,055,403). Enzymes having pullulanase activity used commercially are produced for examples, from Bacillus species (trade name OPTIMAX® L-1000 acid-stable pullulanase from Danisco-Genencor and Promozyme™ pullulanase from Bacillus acidopullulyticus from Novozymes. Bacillus megaterium amylase/transferase (BMA): Bacillus megaterium amylase has the ability to convert the branched saccharides to a form that is easily hydrolyzed by glucoamylase (Hebeda et al., Starch/Starke, 40, 33-36 (1988)). The enzyme exhibits maximum activity at pH 5.5 and temperature at 75° C. (David et al., Starch/Starke, 39 436-440 (1987)). The enzyme has been cloned, expressed in a genetically engineered Bacillus subtilis and produced on a commercial scale (Brumm et al., Starch/Starke, 43 315-329 (1991)). The enzyme is sold under a trade name MEGADEX™

### C. Glucoamylase-Pullulanase Blends

Glucoamylase and pullulanase can be supplied separately or prepackaged as a blend. Such blends are commercially available as OPTIMAX® HDS, or 4060 VHP. OPTIMAX® HDS is 20:80 GAU:ASPU and OPTIMAX 4060 VHP is 40 units GAU and 60 units of ASPU.

### EXAMPLES

### Methods

1. Carbohydrate Composition by High Pressure Liquid Chromatographic (HPLC)

The composition of the reaction products of oligosaccharides was measured by high pressure liquid chromatographic method (Beckman System Gold 32 Karat Fullerton, Calif., USA) equipped with a HPLC column (Rezex RCM-Monosaccharide Ca+ (8%)), maintained at 80° C. fitted with a refractive index (RI) detector (ERC-7515A, RI Detector from The Anspec Company, Inc.). Reverse osmosis (RO) water was used as the mobile phase at a flow rate of 0.6 ml per minute. 20 μL 4.0% solution was injected on to the column. The column separates based on the molecular weight of the saccharides. For example a designation of DP1 is a monosaccharide, such as glucose; a designation of DP2 is a disaccharide, such as maltose; a designation of DP3 is a trisaccharide, such as maltotriose and the designation “DP4⁺” is an oligosaccharide having a degree of polymerization (DP) of 4 or greater.

2. Glucoamylase Activity Units (GAU)

One Glucoamylase Unit is the amount of enzyme that liberates one gram of reducing sugars calculated as glucose from a 2.5% dry substance soluble Lintner starch substrate per hour at 60° C. and 4.3 pH buffered with 20 mM sodium acetate.

3. Pullulanase Activity Units (ASPU)

One Acid Stable Pullulanase Unit (ASPU) is the amount of enzyme which liberates one equivalent reducing potential as glucose per minute from pullulan at pH 4.5 and a temperature of 60° C.

4. Alpha Amylase Activity (AAU)

One AAU of bacterial alpha-amylase activity is the amount of enzyme required to hydrolyze 10 mg of starch per min from 5% dry substance soluble Lintner starch solution containing 31.2 mM calcium chloride, at 60° C. and 6.0 pH buffered with 30 mM sodium acetate.

5. Viscosity Measurement

Measurement of the increase in starch viscosity due to swelling and gelatinization of the granules and the decrease in viscosity as the result of alpha amylases was automated and miniaturized by the use of Newport Instruments SUPER 4 viscometer. This highly automated instrument allows the precise control of temperature rate increase and resulting hydrolysis for the evaluation and characterization of starches, amylases, and various techniques as additives for controlled cooking of starches.

### Example 1

### Viscometer Study for 42% Ds Starch Slurry and its Comparison to 38% Ds Starch Slurry

A series of experiments was performed using an RVA Super 4 (Newport Scientific/Perten Instruments, Huddinge Sweden) to compare high temperature viscographs of 38% ds starch slurry with 42% ds starch slurry. Comparisons were also made with enzyme modified 42% ds starch slurries to compare the peak viscosities.

Two viscograph test profiles were used in this test. The first profile was to test the viscosity variation of the starch slurry at 38 or 42% % ds incubated at 60° C. with constant mixing at 160 rpm for 30 min period of incubation without enzyme addition. The RVA project settings used were: Step 1) Test started at 30° C. with 160 rpm mixing, Step 2) the temperature was maintained at 30° C. for 1 min, Step 3) the slurry was heated to 60° C. in 1 min, Step 4) the slurry was maintained at 60° C. with constant mixing at 160 rpm, and step 5) the slurry was cooled to 30° C. and end the test.

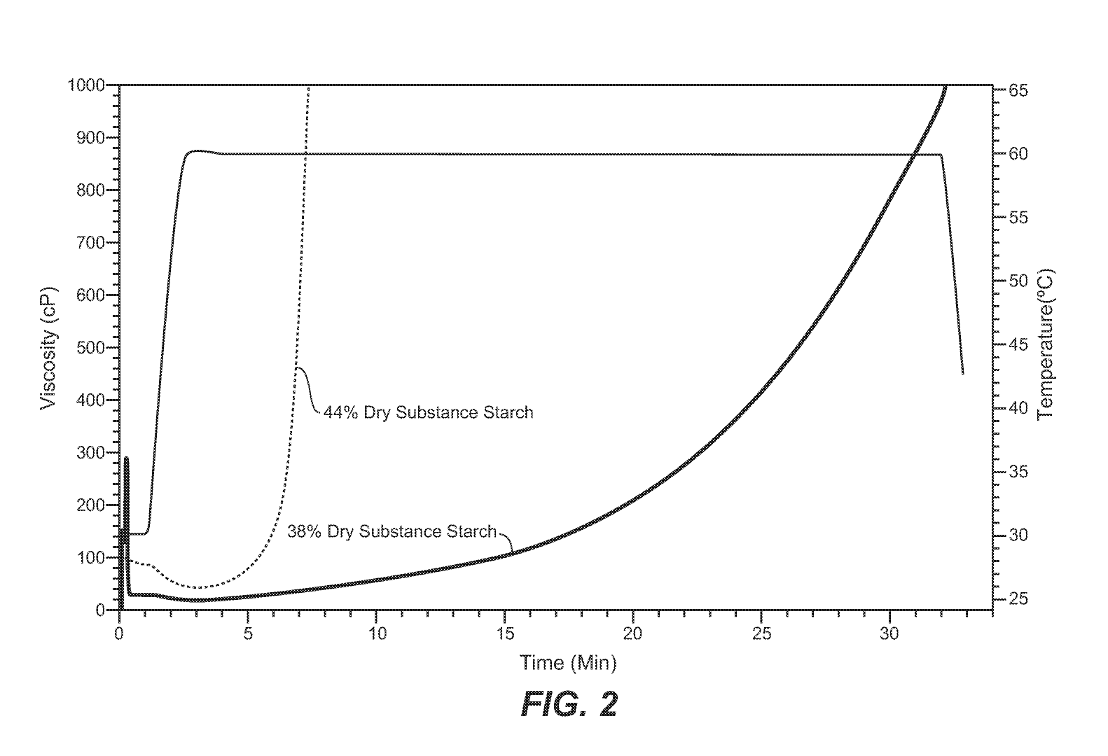

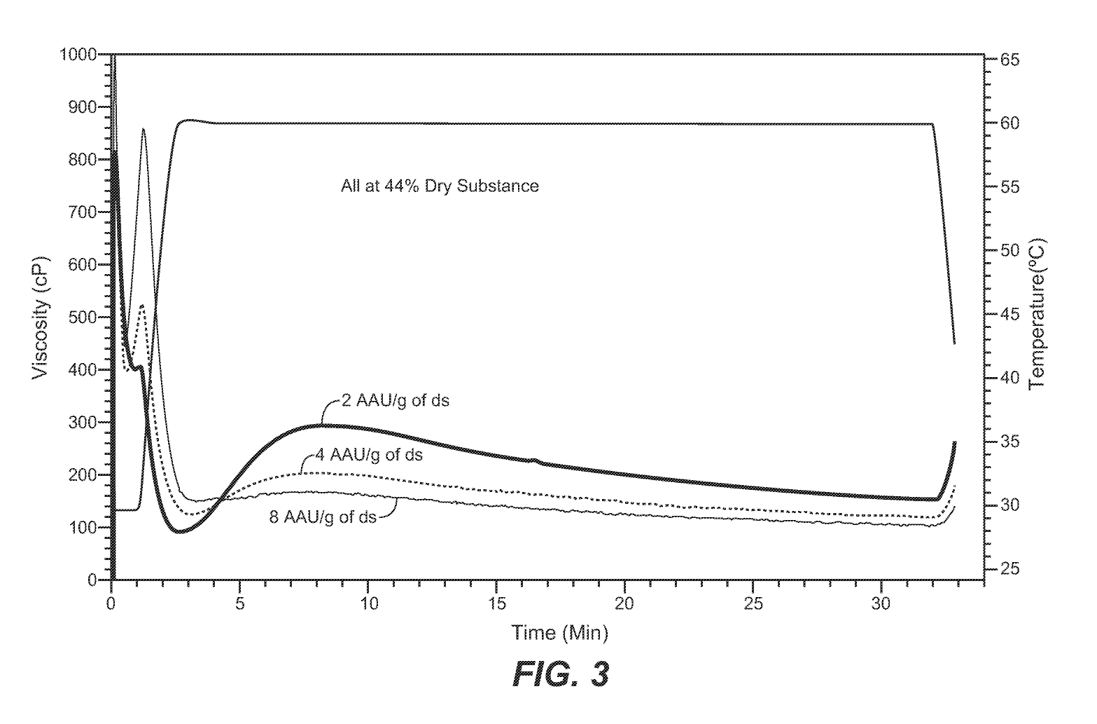

FIG. 2 shows that when the slurry was incubated at 60° C. for 30 min with 160 rpm mixing without any enzyme addition there was a consistent rise in viscosity which was almost exponential. FIG. 3 shows that contacting starch with increasing concentrations of alpha amylase reduced the slurry to less than 200 cP during the 30 min incubation.

### Example 2

### High DS Laboratory Bench Scale Liquefaction

A 1329 g starch slurry was prepared in a 2 liter stainless steel beaker by adding 693 g granular starch (88.15% ds) to 636 g of water to provide 46.4% ds. pH was adjusted to 5.7 with sodium carbonate. The two liter SS beaker was suspended in a 60° C. water bath heated to 60° C. with constant stirring. This was dosed with 0.2 GAU/gdss (0.3093 g) and 4 AAU/gdss (0.1758 g). A sample of the starch slurry was taken after 20 minutes of treatment and found to be 16% soluble with the soluble fraction containing 43% DP1, 18% DP2, 8% DP3 and 31% higher sugars by LC.

After holding 30 min at 60° C. the starch slurry was pumped through a laboratory bench scale cooker consisting of two time delay coils suspended in temperature controlled oil baths. The first coil is a pre-heat coil with about 105 sec of residence time with the second coil being the main cooking coil containing 7-8 minutes of residence time. Temperatures are measured and controlled at the entrance and exit of the cooking coil. For this test the temperature was set at 108.6° C. The temperature in the system was maintained at 15 psi of back pressure using a spring loaded relief valve to enable cooking at >100° C. A 250 ml aliquot of the cooked starch was held at 95° C. to simulate the dextrinization step in commercial liquefaction systems. DE's were determined at 30, 60, 90 and 120 min. The rate of DE development was 0.075 DE per min with a 120 min DE being 21.4.

The 120 min sample was dosed with 0.11 GAU/gdss using OPTIMAX® HDS saccharifying enzyme having a ratio of 20:80 GA:ASPU available from Genencor A Danisco Company. Samples were taken at various times and tested for saccharide distribution by LC and are shown in Table 1

By the time the liquefied starch was prepared for saccharification, the DS by RI was determined to be 48%. The final dry substance by RI at the end of saccharification was 52.1% due to chemical gain from hydrolysis.

TABLE 1

| Hours at | | | | |
|---|---|---|---|---|
| 60° C. | % DP1 | % DP2 | % DP3 | % DP4 |
| 15 | 69.84 | 13.48 | 1.74 | 14.94 |
| 24 | 83.71 | 6.27 | 2.15 | 7.87 |
| 39 | 91.35 | 3.70 | 2.13 | 2.82 |
| 44 | 91.72 | 3.71 | 2.10 | 2.46 |
| 48 | 92.43 | 3.58 | 1.93 | 2.07 |
| 63 | 92.54 | 4.01 | 1.86 | 1.60 |
| 88 | 93.67 | 4.30 | 1.44 | 0.59 |

### Example 3

### High DS Pilot Plant Liquefaction

Liquefaction of 42% dry substance starch slurry was conducted by preparing a slurry containing 25.46 kg of R.O. water and 22.68 kg of Cargill Gel 3420 common dry corn starch. This 23.65° Baumé (corrected) slurry was adjusted by adding 62 g of 6.5% sulfurous acid and 6.5 g of calcium chloride dihydrate which provided 100 ppm SO₂ and 10 ppm calcium. The pH of the slurry was then adjusted to 5.8 using a solution of 20% sodium carbonate.

The slurry was then heated to 60° C. 10 LU/gdss starch of SPEZYME® FRED and 4 AA of SPEZYME® XTRA was added and the temperature was held at 60° C. for 10 minutes to effect viscosity reduction and enable the 42% ds slurry to be pumped to liquefaction. The slurry was then liquefied in a HydroThermal (Waukesha, Wis.) model M-101 steam jet cooker at 106-108° C. for 8 minutes at a back pressure of 16 psi. This material was then held at 95° C. for one hr for further dextrinization. The final liquefact had a DE of 16.6 and was 41.2% dry substance.

This material was adjusted to 4.0 pH with 6 N HCl and held 20 min at 95° C. to terminate remaining alpha amylase activity. The material was then cooled to 60° C. and adjusted to 4.5 pH. Two aliquots were then saccharified with OPTIMAX® 4060 VHP dosed at 0.14 GAU/gdss and OPTIMAX® HDS dosed at 0.11 GAU/gdss.

Table 2 shows the results of the saccharification and confirm that 95% DP1 syrup can be marginally achieved following liquefaction at 42% dry substance by using OPTIMAX HDS which has a ratio of 20:80 GAU:ASPU, while the 40:60 ratio product that is typically used in high glucose production at lower solids does not achieve the >95% target. The ds after liquefaction was 41% due to steam condensation during this step. The final dry substance following saccharification was 45.5% due to chemical gain.

TABLE 2

| | Hr | % DP1 | % DP2 | % DP3 | % DP4+ |
|---|---|---|---|---|---|
| OPTIMAX 4060 | 20 | 84.7 | 5.4 | 1.7 | 8.3 |
| VHP dosed at 0.14 | 28 | 91.4 | 3.0 | 1.8 | 3.9 |
| GAU/gdss | 41 | 94.5 | 2.7 | 1.5 | 1.4 |
| | 48 | 94.8 | 2.7 | 1.3 | 1.2 |
| | 68 | 94.6 | 3.3 | 1.1 | 1.0 |
| OPTIMAX HDS | 20 | 76.3 | 10.3 | 2.0 | 11.4 |
| dosed at 0.11 | 28 | 88.5 | 4.9 | 1.9 | 4.6 |
| GAU/gdss | 41 | 94.2 | 2.6 | 1.7 | 1.4 |
| | 48 | 94.8 | 2.5 | 1.6 | 1.1 |
| | 68 | 95.0 | 2.9 | 1.3 | 0.9 |

### Example 4

### High DS Pilot Plant Liquefaction Using 100% B. stearothermophilus Alpha Amylase

Liquefaction slurry was prepared as described in example 3, except that 8 AAU of SPEZYME® XTRA was used in the hydrolysis. The liquefied slurry was held at 95° C. for 30 min. The final liquefact had a DE of 16.2 and was 41.0% dry substance.

This material was adjusted to 4.0 pH with 6 N HCl and held 20 min at 95° C. to terminate remaining alpha amylase activity. The material was then cooled to 60° C. and adjusted to 4.5 pH. Three aliquots were then saccharified with glucoamylase pullulanase blends of 2.5:97.5, 5:95 and 10:90 at doses of 0.1 GAU/gdss, 0.12 GAU/gdss and 0.14 GAU/gdss respectively. The high ratio was used as shown in example 5 to be preferred (DP1 greater than 95% by weight) and that ratio of 40:60 and 20:80 as shown in example 2 gives lower yields.

Table 3 shows the results of the saccharification and confirmed that >95% DP1 syrup can be produced following liquefaction at 42% dry substance for all doses tested even 70 hours after peak DP1 is achieved. The ds after liquefaction was 41% due to steam condensation during this step. The final dry substance following saccharification was 45.5% due to chemical gain.

TABLE 3

| | Hours | | | | |
|---|---|---|---|---|---|
| Doses | at 60° C. | % DP1 | % DP2 | % DP3 | % DP4+ |
| 2.5:97.5 | 5 | 32.34 | 12.81 | 10.68 | 44.16 |
| GA:Pul ratio at | 19 | 81.38 | 12.91 | 1.23 | 4.47 |
| 0.1 GAU/gdss | 27 | 90.81 | 5.79 | 1.59 | 1.81 |
| | 44 | 95.44 | 2.69 | 1.28 | 0.59 |
| | 74 | 95.70 | 2.98 | 0.90 | 0.42 |
| | 119 | 95.25 | 3.67 | 0.71 | 0.38 |
| 5:95 GA:Pul | 5 | 35.02 | 13.89 | 9.67 | 41.42 |
| ratio at 0.12 | 19 | 83.28 | 10.50 | 1.61 | 4.61 |
| GAU/gdss | 27 | 92.15 | 4.55 | 1.52 | 1.78 |
| | 44 | 95.59 | 2.62 | 1.19 | 0.60 |
| | 74 | 95.62 | 3.09 | 0.85 | 0.43 |
| | 119 | 95.11 | 3.83 | 0.69 | 0.37 |
| 10:90 GA:Pul | 5 | 41.79 | 21.82 | 8.17 | 28.22 |
| ratio at 0.14 | 19 | 88.57 | 5.51 | 1.49 | 4.43 |
| GAU/gdss | 27 | 94.35 | 2.92 | 1.33 | 1.39 |
| | 44 | 95.58 | 2.77 | 1.01 | 0.63 |
| | 74 | 95.25 | 3.51 | 0.76 | 0.48 |
| | 119 | 94.49 | 4.41 | 0.70 | 0.40 |

### Example 5

This example shows saccharification of high dry solids liquefied starch as substrate for producing high density dextrose more than 95.5% using high pullulanase containing GA blend.

32% DS CLEARFLOW® AA liquefied starch was evaporated to increase solids by incubating in a 95° C. water bath to reach 38-42% DS and then pH adjusted to 4.3 with NaOH. Each of 50 g liquefact having 40 and 42% ds was incubated at 60° C. for saccharification by dosing 0.12 GAU/gdss of OPTIDEX® L-400 and 4.68 ASPU/gdss of OPTIMAX® L-1000, a 2.5:97.5 ratio. Reaction was carried out up to 65.5 hr, stopped for periodical sampling by boiling to inactivate enzyme. The boiled samples were diluted by taking 0.5 ml and combining it with 4.5 ml of RO water. This was then filtered through 0.2 m Whatman filters and put into vials for HPLC analysis. The HPLC analysis was conducted using a Rezex RCM-Monosaccharide column.

TABLE 4
Saccharification of High DS

| DS | Hour | % DP1 | % DP2 | % DP3 | % DP4+ |
|---|---|---|---|---|---|
| 40% | 17 | 81.57 | 10.01 | 1.56 | 6.86 |
| | 24 | 91.23 | 4.98 | 1.37 | 2.42 |
| | 40.5 | 95.94 | 2.62 | 0.92 | 0.41 |
| | 48 | 96.01 | 2.49 | 1.00 | 0.50 |
| | 65.5 | 95.81 | 2.84 | 0.90 | 0.46 |
| 42% | 17 | 81.26 | 9.70 | 1.59 | 7.45 |
| | 24 | 90.78 | 5.02 | 1.46 | 2.75 |
| | 40.5 | 95.68 | 2.58 | 1.15 | 0.59 |
| | 48 | 95.81 | 2.66 | 1.04 | 0.49 |
| | 65.5 | 95.52 | 3.04 | 0.96 | 0.49 |

### Example 6

### High DS Pilot Plant Liquefaction Using 100% SPEZYME® XTRA

Liquefaction slurry was prepared as described in example 3, in which 8 AAU/gdss SPEZYME® XTRA was used in place of 10 LU/gdss of SPEZYME® FRED+4 AAU/gdss. The liquefied slurry was held at 95° C. for 30 minutes. The final liquefact had a DE of 15.8 and was 41.2% dry substance.

A control cook using the same SPEZYME® XTRA dose but without the 60° C. treatment resulted in extreme instability of the steam jet cooker due to high viscosity which resulted in erratic combination of the steam and starch slurry. The resulting swings in cooking temperature from 90-120° C. resulted in plugging of the cooker at the inlet.

This material was adjusted to 4.0 pH with 6 N HCl and held 20 min at 95° C. to terminate remaining alpha amylase activity. The material was then cooled to 60° C. and adjusted to 4.5 pH. Five aliquots were then saccharified with A. niger glucoamylase/pullulanase blends of 2.5:97.5, 5:95 and 10:90, 20:80 and 40:60 at doses of 0.1 GAU/gdss, 0.12 GAU/gdss 0.14, 0.15 and 0.16 GAU/gdss respectively. The high ratio was used as shown in example 5 and was found to be preferred (DP1 greater than 95%) and that ratios of 40:60 and 20:80 as shown in example 2 gave lower yields.

Table 5 shows the results of the saccharification and confirms that >95% DP1 syrup can be produced following liquefaction at 42% dry substance with minimum reversion resulting in >95% DP1 which remained steady even after peak DP1 is achieved for the case of 2.5:97.5, 5:95 and 10:90 ratios. The ds after liquefaction was 41% due to steam condensation during this step. The final dry substance following saccharification was 45.5% due to chemical gain.

TABLE 5

| | Hours | | | | |
|---|---|---|---|---|---|
| Doses | at 60° C. | % DP1 | % DP2 | % DP3 | % DP4+ |
| 2.5:97.5 GA:Pul | 18 | 73.84 | 16.00 | 1.89 | 8.26 |
| ratio at 0.1 | 24 | 84.21 | 10.26 | 1.78 | 3.76 |
| GAU/g ds | 42 | 94.45 | 3.12 | 1.49 | 0.93 |
| | 48 | 95.04 | 2.78 | 1.42 | 0.76 |
| | 54 | 95.20 | 2.74 | 1.31 | 0.74 |
| 5:95 GA:Pul | 18 | 80.90 | 11.25 | 1.73 | 6.12 |
| ratio at 0.12 | 24 | 89.79 | 6.01 | 1.66 | 2.54 |
| GAU/g ds | 42 | 95.15 | 2.77 | 1.29 | 0.78 |
| | 48 | 95.23 | 2.84 | 1.22 | 0.71 |
| | 54 | 95.30 | 2.89 | 1.13 | 0.69 |
| 10:90 GA:Pul | 18 | 84.97 | 7.30 | 1.59 | 6.14 |
| ratio at 0.14 | 24 | 91.90 | 3.93 | 1.54 | 2.63 |
| GAU/g ds | 42 | 95.18 | 2.84 | 1.11 | 0.87 |
| | 48 | 95.14 | 2.98 | 1.10 | 0.79 |
| | 54 | 95.24 | 3.02 | 0.99 | 0.74 |
| 20:80 GA:Pul | 18 | 84.99 | 5.29 | 1.44 | 8.29 |
| ratio at 0.15 | 24 | 91.05 | 3.31 | 1.40 | 4.24 |
| GAU/g ds | 42 | 94.59 | 2.99 | 1.14 | 1.27 |
| | 48 | 94.78 | 3.09 | 1.04 | 1.08 |
| | 54 | 94.93 | 3.13 | 0.95 | 0.99 |
| 40:60 GA:Pul | 18 | 85.58 | 3.96 | 1.26 | 9.19 |
| ratio at 0.16 | 24 | 90.74 | 2.96 | 1.25 | 5.05 |
| GAU/g ds | 42 | 94.18 | 3.11 | 1.07 | 1.64 |
| | 48 | 94.54 | 3.19 | 0.94 | 1.33 |
| | 54 | 94.59 | 3.32 | 0.90 | 1.19 |

### Example 7

### High DS Pilot Plant Liquefaction Using 100% SPEZYME® XTRA Followed by Saccharification with Humicola Glucoamylase (HGA)

The liquefaction slurry was prepared as described in Example 3, in which 8 AAU of SPEZYME® XTRA was used in the hydrolysis. The liquefied slurry was held at 95° C. for 30 min. The final liquefact had a DE of 13.4 and was 41.2% dry substance.

Three aliquots of this material were retained. One was adjusted to 4.0 pH with 6 N HCl and held 20 min at 95° C. to terminate remaining alpha amylase activity. The second was heated to 130° C. for 7 min to terminate the remaining alpha amylase activity, and the third aliquot was used as is with the alpha amylase activity allowed to remain. The material was then cooled to 60° C. and adjusted to 5.5 pH. The aliquots were then saccharified with H. grisea glucoamylase/pullulanase blends of 5:95 at doses 0.12 GAU/gdss. The high ratio was used as shown in example 5 and was found to give greater than 95% DP1 as shown in Table 5.

The results shown in Table 6 show that HGA with added pullulanase will produce >95% DP1 when the alpha amylase activity is terminated via acid kill. Substrate with heat kill treatment was 0.1% DP1 lower than the target. Leaving the liquefaction alpha amylase active during saccharification depresses the DP 1 achievement by about 0.6% DP 1. This difference is found in the DP 3 region.

TABLE 6
All saccharification done with 0.12 GAU/g from HGA
with GA:Pullulanase ratio of 5:95

| | Hours at | | | | |
|---|---|---|---|---|---|
| | 60° C. | DP1 | DP2 | DP3 | DP4+ |
| Acid Killed AA | 6 | 41.26 | 15.05 | 9.37 | 34.33 |
| liquefied starch | 17 | 78.73 | 11.76 | 1.51 | 8.00 |
| | 26 | 88.96 | 6.22 | 1.67 | 3.16 |
| | 49 | 95.33 | 3.05 | 1.09 | 0.54 |
| | 66 | 95.01 | 3.33 | 0.88 | 0.77 |
| Heat killed AA | 6 | 43.81 | 14.80 | 7.11 | 34.28 |
| liquefied starch | 17 | 78.70 | 9.79 | 1.36 | 10.16 |
| | 26 | 88.28 | 5.62 | 1.50 | 4.60 |
| | 49 | 94.89 | 3.00 | 1.05 | 1.06 |
| | 66 | 93.82 | 3.98 | 0.91 | 1.28 |
| Active AA liquefied | 6 | 43.09 | 21.38 | 11.61 | 23.91 |
| starch | 17 | 76.97 | 15.78 | 2.29 | 4.96 |
| | 26 | 90.04 | 8.05 | 0.83 | 1.07 |
| | 49 | 94.69 | 3.20 | 1.63 | 0.48 |
| | 66 | 94.27 | 3.46 | 1.22 | 1.05 |

All patents and publications, including all sequences disclosed within such patents and publications, referred to herein are expressly incorporated by reference in their entirety for all purposes. Insofar as the product referred to by a trademark name varies with time, the product having the characteristics described in the relevant product literature, including websites, from the manufacturer as of the effective filing date of the application is intended. Such product literature is also incorporated by reference in its entirety for all purposes. The headings provided herein are not limitations of the various aspects or embodiments of the invention, which can be had by reference to the specification as a whole. Although preferred methods and materials have been described, any methods and materials similar or equivalent to those described herein can be used in the practice or testing of the present invention. Unless otherwise apparent from the context, any embodiment, aspect, step, feature, element or limitation can be used in combination with any other.

What is claimed is:

1. A method of processing granular starch comprising:
(a) contacting granular starch, water and one or more granular starch hydrolyzing enzymes including an alpha-amylase and/or a glucoamylase to produce slurry in which the concentration of dry solids is greater than 38% by weight,
(b) incubating the slurry at a temperature above 40° C. and at or below the gelatinization temperature of the granular starch for at least five minutes to produce a composition in which the granular starch has been partially hydrolyzed into oligo- and/or mono-saccharides by the one or more enzymes; and
(c) raising and holding the temperature of the partially hydrolyzed composition above the gelatinization temperature of the granular starch to produce a liquefied composition.

2. The method of claim 1, further comprising (d) contacting the liquefied composition with pullulanase and glucoamylase and incubating to produce glucose.

3-4. (canceled)

5. The method of claim 1, wherein the percentage of dry solids is at least 39% throughout steps (a)-(d).

6. (canceled)

7. The method of claim 1, wherein the percentage of dry solids remains the same or increases between steps (a) and (d).

8. The method of claim 2, wherein no more than 10% by weight water is added to the slurry throughout steps (b), (c) and (d).

9. The method of claim 2, wherein no more water is added to the slurry throughout steps (b), (c) and (d).

10. The method of claim 1, wherein the one or more enzymes includes one or more alpha amylase.

11. The method of claim 10, wherein the alpha amylase is an alpha amylase selected from the group consisting of SPEZYME® AA, SPEZYME® XTRA®, SPEZYME® FRED, GYZME® G997, TERMAMYL®, 120-L, LC, SC, SUPRA or Fuelzyme®.

12-13. (canceled)

14. The method of claim 10, wherein the alpha amylase is thermostable and remains active in step (c).

15. The method of claim 1, wherein the temperature in step (b) is 55-67° C.

16. (canceled)

17. The method of claim 1, wherein the temperature of step (c) is 90-110° C.

18. The method of claim 17, wherein the composition is held at 90-110° C. for 5 min to 4 hours.

19. The method of claim 17, wherein the composition is held at 100-110° C. for 5-20 minutes and at 90-100° C. for 1-2 hr.

20. The method of claim 2, wherein the ratio of pullulanase to glucoamylase in step (d) is at least 9:1 by units.

21. The method of claim 2, wherein step (d) is performed at a temperature of 40-80° C.

22. The method of claim 21, wherein step (d) is performed for 20-150 hours.

23-24. (canceled)

25. The method of claim 1, wherein the one or more enzymes includes an alpha-amylase and the method further comprises deactivating the alpha amylase after step (c) by heat or acid treatment.

26-27. (canceled)

28. The method of claim 2, wherein the pH is between 4.9 and 5.5 throughout steps (b), (c) and (d).

29. (canceled)

30. The method of claim 2, wherein the pullulanase in step (d) is from Bacillus and the glucoamylase is from Aspergillus niger or Humicola grisea.

31-32. (canceled)

33. The method of claim 1, wherein the one or more enzyme are one or more alpha amylases and the method further comprises allowing the liquefied composition to cool, whereby the one or more alpha amylases of step (a) or one or more fresh alpha amylases hydrolyze starch in the liquefied composition to oligosaccharides producing a maltodextrin composition.

34-36. (canceled)
